(19) United States
(12) Patent Application Publication
(10) Pub. No.: US 2026/0028636 A1
(43) Pub. Date: Jan. 29, 2026

(54) TRANSCRIPTION REGULATING NUCLEOTIDE SEQUENCES AND METHODS OF USE

(71) Applicant: BASF Agricultural Solutions Seed US LLC, Research Triangle Park, NC (US)

(72) Inventors: Erin Marie Davis, Research Triangle Park, NC (US); Sebastian Hermann Martschat, Ludwigshafen (DE); Jonathan T. Vogel, Research Triangle Park, NC (US); Hunter James Cameron, Research Triangle Park, NC (US); Zhixin Shi, Research Triangle Park, NC (US)

(73) Assignee: BASF Agricultural Solutions Seed US LLC, Research Triangle Park, NC (US)

(21) Appl. No.: 18/705,253

(22) PCT Filed: Oct. 27, 2022
(86) PCT No.: PCT/US2022/078751

§ 371 Date: Apr. 26, 2024

Related U.S. Application Data
(60) Provisional application No. 63/272,492, filed on Oct. 27, 2021.

(51) Int. Cl. C12N 15/82; C12N 1/20; C12N 5/04; G16B 20/20; G16B 30/10

(52) U.S. Cl. CPC C12N 15/8216; C12N 1/20; C12N 5/04; C12N 15/8202; C12N 15/8274; C12N 15/8286; G16B 20/20; G16B 30/10; C12N 2830/001

(57) ABSTRACT

Described herein are transcription regulating nucleotide sequences and the use of such transcription regulating nucleotide sequences to express a polynucleotide of interest in plants.

### FIELD OF THE INVENTION

Described herein are transcription regulating nucleotide sequences and the use of such transcription regulating nucleotide sequences to express a polynucleotide of interest in plants as well as methods of identifying and optimizing such regulating nucleotide sequences.

### BACKGROUND

Modification of plants to alter and/or improve phenotypic characteristics (such as productivity or quality) requires the overexpression or down-regulation of endogenous genes or the expression of heterologous genes in plant tissues. Such genetic modification relies on the availability of a means to drive and to control gene expression as required. Indeed, genetic modification relies on the availability and use of suitable promoters and motifs which are effective in plants and which regulate gene expression so as to give the desired effect(s) in the transgenic plant. Also what is needed are methods that can be used to efficiently identify and/or optimize such promoters and motifs to allow for efficient expression of transgenes in plants.

### SUMMARY

In one aspect, described herein is an expression cassette for regulating expression of a polynucleotide of interest, said expression cassette comprising a transcription regulating nucleotide sequence that is at least 60% identical to the nucleic acid sequence set forth in SEQ ID NO: 1, SEQ ID NO: 2, SEQ ID NO: 3, SEQ ID NO: 4, SEQ ID NO: 5, SEQ ID NO: 6 or a functional fragment thereof. In some embodiments, the transcription regulating nucleotide sequence is at least 65% (or at least 70%, 75%, 80%, 81%, 82%, 83%, 84%, 85%, 96%, 97%, 88%, 89%, 90%, 91%, 92%, 93%, 94%, 95%, 96%, 97%, 98%, or at least 99%) or more identical to the nucleotide sequence set forth in SEQ ID NO: 1, SEQ ID NO: 2, SEQ ID NO: 3, SEQ ID NO: 4, SEQ ID NO: 5, or SEQ ID NO: 6. In some embodiments, the transcription regulating nucleotide sequence comprises the nucleic acid sequence set forth in SEQ ID NO: 1, SEQ ID NO: 2, SEQ ID NO: 3, SEQ ID NO: 4, SEQ ID NO: 5, or SEQ ID NO: 6.

The expression cassette in some embodiments further comprises at least one polynucleotide of interest being operatively linked to the transcription regulating nucleotide sequence. In some embodiments, the polynucleotide of interest is an herbicide-tolerance coding sequence, an insecticidal coding sequence, an nematicidal coding sequence, an antimicrobial coding sequence, an antifungal coding sequence, an antiviral coding sequence, an abiotic and biotic stress tolerance coding sequences, or a sequence modifying plant traits such as yield, grain quality, nutrient content, starch quality and quantity, nitrogen fixation and/or utilization, and oil content, sequence modifying plant size, height, structure or architecture, and/or composition. In some embodiments, the polynucleotide of interest is heterologous with respect to the transcription regulating nucleotide sequence.

In another aspect, the disclosure provides a vector comprising an expression cassette described herein. In some embodiments, the vector is an expression vector.

In another aspect, the disclosure provides a host cell comprising an expression cassette or vector described herein. In some embodiments, the host cell is a plant cell.

In another aspect, the disclosure provides a transgenic plant tissue, plant organ, plant or seed comprising an expression cassette or a vector described herein. In some embodiments, the transgenic plant tissue, plant organ, plant or seed is a monocotyledonous plant tissue, plant organ, plant or seed. In some embodiments, the transgenic plant tissue, plant organ, plant or seed is a dicotyledonous plant tissue, plant organ, plant or seed. In some embodiments, the transgenic plant tissue, plant organ, plant or seed is hemizygous for the expression cassette. In some embodiments, the transgenic plant tissue, plant organ, plant or seed is homozygous for the expression cassette.

In another aspect, the disclosure provides a method for expressing a polynucleotide of interest in a host cell comprising (a) introducing an expression cassette or a vector of described herein into the host cell, and (b) expressing at least one polynucleotide of interest in said host cell. In some embodiments, the host cell is a plant cell. In some embodiments, the detectable amount of protein accumulated that is encoded by the polynucleotide of interest is about 0.01%-1.15% (or about 0.05%-1.15%, or about 0.1%-1.15%, or about 0.5%-1.15%, or about 1%-1.15%) of the extracted total soluble proteins. The term “Total Soluble Protein (TSP)” as used herein refers to all proteins able to be solubilized in a buffer suitable for protein quantification typically facilitated by mechanical disruption.

In another aspect, the disclosure provides a method for producing a transgenic plant tissue, plant organ, plant or seed comprising (a) introducing an expression cassette or a vector described herein into a plant cell; and (b) regenerating said plant cell to form a plant tissue, plant organ, plant or seed. In some embodiments, the method further comprises selecting the plant cell to form a plant tissue, plant organ, plant or seed for the presence of the expression cassette or the vector. In some embodiments, two or more copies of the expression cassette are introduced into the plant cell.

In another aspect, the disclosure provides a method of providing pesticidal activity in a plant comprising (a) introducing the expression cassette comprising a polynucleotide sequence that encodes a pesticidal protein into a host cell of the plant, and (b) expressing the polynucleotide that encodes a pesticidal protein in said host cell, thereby providing pesticidal activity in the plant. In some embodiments, the pesticidal protein is an insecticidal protein. In some embodiments, two or more copies of the expression cassette are introduced into the plant cell.

In another aspect, the disclosure provides methods of identifying a transcription regulating polynucleotide sequence by detecting the presence of the sequence GATCTG in a nucleotide sequence upstream to a coding sequence (interchangeably the, “Motif” or “k-mer” or “GATCTG”). Applicants used the methods as described in publication WO 2022/098588 herein incorporated in entirety by reference, to identify the Motif as significantly associated with transcription regulating polynucleotide sequences able to be utilized in an expression vector to constitutively express transgenes in a plant. Applicants, based on this observation and by way of example, were able to identify three native plant nucleotide sequences containing the Motif that can be utilized as a transcription regulating polynucleotide sequence (e.g. SEQ ID NOs: 2, 3 and 5).

In another aspect, the disclosure provides methods to increase the efficiency of a transcription regulating polynucleotide sequence. Particularly, it was surprisingly found that when the Motif is removed in 1, 2, 3 or more instances, the performance of the transcription regulating polynucleotide sequence improves as further shown below. Not to be limited by theory, it is also contemplated that the editing (i.e. gene editing) of the Motif sequence itself could lead to the same beneficial results as observed. For example, one could edit any nucleotide base in GATCTG to any alternative nucleotide base.

### BRIEF DESCRIPTION OF THE FIGURES

FIG. 1 shows the expression performance of UBC-m promoter as depicted in SEQ ID NO: 1 and UBC-n promoter as depicted in SEQ ID NO: 2 against a control driving luciferase expression in a transient tobacco leaf assay. The native and mutant forms of UBC result in high expression of luciferase compared to the positive control, Ubiquitin (UBQ10). The negative, uninfiltrated control is not shown, as the values were close to zero and did not allow proper scaling for the promoter data. A mutated promoter with all occurrences of the k-mer GATCTG (UBC-mutated, SEQ ID NO: 1) removed showed reduced variation in expression compared to the native sequences.

FIG. 2 shows expression of promoters CSI1 and TMN12 with and without the k-mer GATCTG driving luciferase expression in a transient tobacco leaf assay. Respectfully TMN12 native sequence SEQ ID NO: 3; TMN12 mutant sequence SEQ ID NO: 4; CSI1 native sequence SEQ ID NO: 5 and CSI1 mutant sequence SEQ ID NO: 6. The native and mutant forms of the TMN12 and CSI1 promoters result in expression of luciferase compared to the negative, uninfiltrated control. Mutated promoters with all occurrences of the k-mer GATCTG removed showed reduced variation in expression compared to the native promoter sequence.

FIG. 3 shows expression of the UBC-n promoter in transformed soybean against a control. QRT-PCR was used to measure AHAS transcript levels in soybean (cv Thorne) seedling leaves. For comparison, a positive control promoter (Super, described in Examples) was used to also drive expression of AHAS. At least 25 independent tranformants were generated and assayed per promoter.

FIG. 4 depicts the relative position of the GATCTG k-mers present in each promoter tested. The relative position of the GATCTG k-mer is indicated by black boxes. The regions depicted represent 1000 bp upstream of the start codon of each of the three genes indicated.

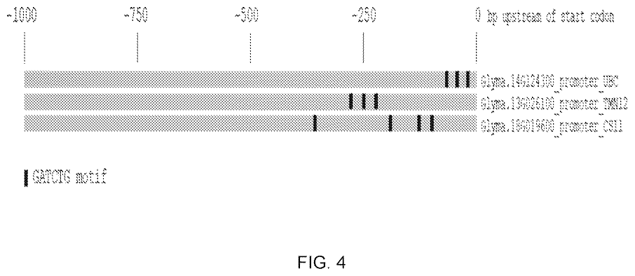

### BRIEF DESCRIPTION OF SEQUENCES

SEQ ID NO: 1 is the UBC mutant promoter with removed Motif sites.

SEQ ID NO: 2 is the native UBC promoter sequence.

SEQ ID NO: 3 is the TMN12 mutant promoter with removed Motif sites.

SEQ ID NO: 4 is the native TMN12 promoter sequence.

SEQ ID NO: 5 is the native CSI1 promoter sequence.

SEQ ID NO: 6 is the CSI1 mutant promoter with removed Motif sites.

### DETAILED DESCRIPTION

The present disclosure provides an expression cassette comprising a transcription regulating polynucleotide sequence that directs constitutive transcription/expression of an operably linked polynucleotide of interest in a plant cell, plant, or plant part. The present invention is based on the discovery that the transcription regulating polynucleotide sequence comprising the nucleic acid sequence set forth in SEQ ID NO: 1, SEQ ID NO: 2, SEQ ID NO: 3, SEQ ID NO: 4, SEQ ID NO: 5, or SEQ ID NO: 6 has constitutive promoter activity in plants.

As used herein, “transcription regulating nucleotide sequence” refers to a nucleotide sequences that influences the transcription, RNA processing or stability, or translation of the associated (or functionally linked) nucleotide sequence to be transcribed. The transcription regulating nucleotide sequence may have various localizations with the respect to the nucleotide sequences to be transcribed. The transcription regulating nucleotide sequence may be located upstream (5′ non-coding sequences), within, or downstream (3′ non-coding sequences) of the sequence to be transcribed (e.g., a coding sequence). The transcription regulating nucleotide sequences may be selected from the group comprising enhancers, promoters, translation leader sequences, introns, 5′-untranslated sequences, 3′-untranslated sequences, and polyadenylation signal sequences. They include natural and synthetic sequences as well as sequences that are a combination of synthetic and natural sequences. As is noted above, the term “transcription regulating nucleotide sequence” is not limited to promoters. However, preferably a transcription regulating nucleotide sequence of the invention comprises at least one promoter sequence (e.g., a sequence localized upstream of the transcription start of a gene capable to induce transcription of the downstream sequences). In one preferred embodiment the transcription regulating nucleotide sequence of the invention comprises the promoter sequence of the corresponding gene and—optionally and preferably—the native 5′-untranslated region of said gene. Furthermore, the 3′-untranslated region and/or the polyadenylation region of said gene may also be employed.

The term “functional fragment thereof” as used herein refers to a nucleic acid sequence that is shorter in length than the transcription regulating nucleotide sequence yet retains the activity of the transcription regulating nucleotide sequence. For example, in some embodiments, the functional fragment of the transcription regulating nucleotide sequences comprises a nucleotide sequence at least 50 bp (or at least 100 bp, at least 150 bp, at least 200 bp, at least 250 bp, at least 300 bp, at least 350 bp, at least 400 bp, at least 450, at least 500 bp, at least 550 bp, at least 600 bp, at least 650 bp, at least 700 bp, at least 750 bp, at least 800 bp, at least 850 bp, at least 900 bp or at least 1000 bp) in length and retains the activity of the transcription regulating nucleotide sequence.

### Expression Vectors

Another object of the present invention refers to a vector comprising the expression cassette of the present invention.

The term “vector”, preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotides of the present invention may comprise selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques well known to those skilled in the art. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, natural competence, carbon-based clusters, chemically mediated transfer, electroporation or particle bombardment (e.g., “gene-gun”). Suitable methods for the transformation or transfection of host cells, including plant cells, can be found in Sambrook et al. (Molecular Cloning: A Laboratory Manual, 2nd ed., Cold Spring Harbor Laboratory, Cold Spring Harbor Laboratory Press, Cold Spring Harbor, NY, 1989) and other laboratory manuals, such as Methods in Molecular Biology, 1995, Vol. 44, Agrobacterium protocols, Ed.: Gartland and Davey, Humana Press, Totowa, New Jersey. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells.

Preferably, the vector referred to herein is suitable as a cloning vector, i.e. replicable in microbial systems. Such vectors ensure efficient cloning in bacteria and, preferably, yeasts or fungi and make possible the stable transformation of plants. Those which must be mentioned are, in particular, various binary and co-integrated vector systems which are suitable for the T DNA-mediated transformation. Such vector systems are, as a rule, characterized in that they contain at least the vir genes, which are required for the Agrobacterium-mediated transformation, and the sequences which delimit the T-DNA (T-DNA border). These vector systems, preferably, also comprise further cis-regulatory regions such as promoters and terminators and/or selection markers with which suitable transformed host cells or organisms can be identified. While co-integrated vector systems have vir genes and T DNA sequences arranged on the same vector, binary systems are based on at least two vectors, one of which bears vir genes, but no T-DNA, while a second one bears T DNA, but no vir gene. As a consequence, the last-mentioned vectors are relatively small, easy to manipulate and can be replicated both in E. coli and in Agrobacterium. An overview of binary vectors and their use can be found in Hellens et al, Trends in Plant Science (2000) 5, 446-451. Furthermore, by using appropriate cloning vectors, the expression cassette of the invention can be introduced into host cells or organisms such as plants or animals and, thus, be used in the transformation of plants, such as those which are published, and cited, in: Plant Molecular Biology and Biotechnology (CRC Press, Boca Raton, Florida), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205 225.

More preferably, the vector of the present invention is an expression vector. In such an expression vector, the expression cassette comprises a transcription regulating nucleotide sequence as specified above allowing for expression in eukaryotic cells or isolated fractions thereof. An expression vector may, in addition to the expression cassette of the invention, also comprise further regulatory elements including transcriptional as well as translational enhancers. Preferably, the expression vector is also a gene transfer or targeting vector. Expression vectors derived from viruses such as retroviruses, vaccinia virus, adeno-associated virus, herpes viruses, or bovine papilloma virus, may be used for delivery of the expression cassettes or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N. Y. and Ausubel, Current Protocols in Molecular Biology, Green Publishing Associates and Wiley Interscience, N.Y. (1994).

Suitable expression vector backbones are, preferably, derived from expression vectors known in the art such as Okayama-Berg cDNA expression vector pcDV1 (Pharmacia), pCDM8, pRc/CMV, pcDNA1, pcDNA3 (Invitrogen) or pSPORT1 (GIBCO BRL). Further examples of typical fusion expression vectors are pGEX (Pharmacia Biotech Inc; Smith, D. B., and Johnson, K. S. (1988) Gene 67:31-40), pMAL (New England Biolabs, Beverly, MA) and pRIT5 (Pharmacia, Piscataway, NJ), where glutathione Stransferase (GST), maltose E-binding protein and protein A, respectively, are fused with the nucleic acid of interest encoding a protein to be expressed. The target gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a coexpressed viral RNA polymerase (T7 gn1). This viral polymerase is provided by the host strains BL21 (DE3) or HMS174 (DE3) from a resident λ-prophage which harbors a T7 gn1 gene under the transcriptional control of the lacUV 5 promoter. Examples of vectors for expression in the yeast S. cerevisiae comprise p YepSecl (Baldari et al. (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, CA). Vectors and processes for the construction of vectors which are suitable for use in other fungi, such as the filamentous fungi, comprise those which are described in detail in: van den Hondel, C.A.M.J.J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23.

In some embodiments, the vector (or vectors) described herein comprising the expression cassette are propagated and amplified in a suitable organism, i.e. expression host. In some embodiments, one copy of the vector is propagated and amplified in a suitable organism. In some embodiments, two or more (e.g., 3, 4, 5, 6 7, 8 or more) copies of the vector are propagated and amplified in a suitable organism.

The term “expression cassette” as used herein refers to a linear or circular nucleic acid molecule. It encompasses DNA as well as RNA sequences which are capable of directing expression of a particular nucleotide sequence in an appropriate host cell. In general, it comprises a promoter operably linked to a polynucleotide of interest, which is—optionally—operably linked to termination signals and/or other regulatory elements. The expression cassette of the present invention is characterized in that it shall comprise a transcription regulating nucleotide sequence as defined hereinafter. An expression cassette may also comprise sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the polynucleotide sequence of interest may be chimeric, meaning that at least one of its components is heterologous with respect to at least one of its other components. The expression cassette may also be one, which is naturally occurring but has been obtained in a recombinant form useful for heterologous expression. An expression cassette may be assembled entirely extracellularly (e.g., by recombinant cloning techniques). However, an expression cassette may also be assembled using in part endogenous components. For example, an expression cassette may be obtained by placing (or inserting) a promoter sequence upstream of an endogenous sequence, which thereby becomes functionally linked and controlled by said promoter sequences. Likewise, a nucleic acid sequence to be expressed may be placed (or inserted) downstream of an endogenous promoter sequence thereby forming an expression cassette. In a preferred embodiment, such expression cassettes will comprise a transcriptional initiation region linked to a nucleotide sequence of interest. Such an expression cassette is preferably provided with a plurality of restriction sites for insertion of the gene of interest to be under the transcriptional regulation of the regulatory regions. The expression cassette may additionally contain selectable marker genes. The cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the Ti-plasmid of A. tumefaciens, such as the octopine synthase and nopaline synthase termination regions and others described below (see also, Guerineau 1991; Proudfoot 1991; Sanfacon 1991; Mogen 1990; Munroe 1990; Ballas 1989; Joshi 1987). The expression cassette can also comprise a multiple cloning site. In such a case, the multiple cloning site is, preferably, arranged in a manner as to allow for operative linkage of a polynucleotide to be introduced in the multiple cloning site with the transcription regulating sequence. In addition to the aforementioned components, the expression cassette of the present invention, preferably, could comprise components required for homologous recombination, i.e. flanking genomic sequences from a target locus. However, also contemplated is an expression cassette which essentially consists of the transcription regulating nucleotide sequence, as defined hereinafter.

The terms “operably-linked” or “functionally linked” refer to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

The term “promoter” as used herein refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised, in some cases, of a TATA box and other sequences that serve to specify the site of transcription initiation, to which regulatory elements are added for enhancement of expression. “Promoter” also refers to a nucleotide sequence that includes a minimal promoter plus regulatory elements and that is capable of controlling the expression of a coding sequence or functional RNA. This type of promoter sequence consists of proximal and more distal upstream elements, the latter elements often referred to as enhancers. Accordingly, an “enhancer” is a DNA sequence, which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements, derived from different promoters found in nature, or even be comprised of synthetic DNA segments.

A promoter may also contain DNA sequences that are involved in the binding of protein factors, which control the effectiveness of transcription initiation in response to physiological or developmental conditions. The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative. Promoter elements, such as a TATA element, that are inactive or have greatly reduced promoter activity in the absence of upstream activation are referred as “minimal” or “core” promoters. In the presence of a suitable transcription factor, the minimal promoter functions to permit transcription. A “minimal” or “core” promoter thus consists only of all basal elements needed for transcription initiation, e.g., a TATA box and/or an initiator.

The term “constitutive promoter” as used herein refers to a promoter that is able to express the open reading frame (ORF) in all or nearly all of the plant tissues during all or nearly all developmental stages of the plant. Each of the transcription-activating elements do not exhibit an absolute tissue-specificity, but mediate transcriptional activation in most plant tissues at a level of at least 1% reached in the plant tissue in which transcription is most active. “Constitutive expression” refers to expression using a constitutive promoter.

The term “regulated promoter” as used herein refers to promoters that direct gene expression not constitutively, but in a temporally—and/or spatially—regulated manner, and includes both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al. (1989). Typical regulated promoters useful in plants include but are not limited to safener-inducible promoters, promoters derived from the tetracycline-inducible system, promoters derived from salicylate-inducible systems, promoters derived from alcohol-inducible systems, promoters derived from glucocorticoid-inducible system, promoters derived from pathogen-inducible systems, and promoters derived from ecdysone-inducible systems. “Conditional” and “regulated expression” refer to expression controlled by a regulated promoter.

“Inducible promoter” refers to those regulated promoters that can be turned on in one or more cell types by an external stimulus, such as a chemical, light, hormone, stress, or a pathogen.

As used herein, the term “cis-regulatory element” or “promoter motif” refers to a cis-acting transcriptional regulatory element that confers an aspect of the overall control of gene expression. A cis-element may function to bind transcription factors, trans-acting protein factors that regulate transcription. Some cis-elements bind more than one transcription factor, and transcription factors may interact in different affinities with more than one cis-element. The promoters of the present invention desirably contain cis-elements that can confer or modulate gene expression. Cis-elements can be identified by a number of techniques, including deletion analysis, i.e., deleting one or more nucleotides from the 5′ end or internal of a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis with known cis-element motifs by conventional DNA sequence comparison methods. The fine structure of a cis-element can be further studied by mutagenesis (or substitution) of one or more nucleotides or by other conventional methods. Cis-elements can be obtained by chemical synthesis or by isolation from promoters that include such elements, and they can be synthesized with additional flanking nucleotides that contain useful restriction enzyme sites to facilitate subsequence manipulation.

### Expression in a Host Cell

In another aspect, described herein is a method for expressing a polynucleotide of interest in a host cell comprising introducing an expression cassette or vector described herein into the host cell and expressing the polynucleotide of interest in the host cell.

The term “expression” as used herein refers to the transcription and/or translation of an endogenous gene, ORF or portion thereof, or a transgene in plants. For example, in the case of antisense constructs, expression may refer to the transcription of the antisense DNA only. In addition, expression refers to the transcription and stable accumulation of sense (mRNA) or functional RNA. Expression may also refer to the production of protein.

The “expression pattern” of a promoter (with or without enhancer) is the pattern of expression levels, which shows where in the plant and in what developmental stage transcription is initiated by said promoter. Expression patterns of a set of promoters are said to be complementary when the expression pattern of one promoter shows little overlap with the expression pattern of the other promoter. The level of expression of a promoter can be determined by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore, the steady state level is the product of synthesis rates and degradation rates. The rate of degradation can however be considered to proceed at a fixed rate when the transcribed sequences are identical, and thus this value can serve as a measure of synthesis rates. When promoters are compared in this way, techniques available to those skilled in the art are hybridization S1-RNAse analysis, northern blots and competitive RT-PCR. This list of techniques in no way represents all available techniques, but rather describes commonly used procedures used to analyze transcription activity and expression levels of mRNA. The analysis of transcription start points in practically all promoters has revealed that there is usually no single base at which transcription starts, but rather a more or less clustered set of initiation sites, each of which accounts for some start points of the mRNA. Since this distribution varies from promoter to promoter the sequences of the reporter mRNA in each of the populations would differ from each other. Since each mRNA species is more or less prone to degradation, no single degradation rate can be expected for different reporter mRNAs. It has been shown for various eukaryotic promoter sequences that the sequence surrounding the initiation site (‘initiator’) plays an important role in determining the level of RNA expression directed by that specific promoter. This also includes part of the transcribed sequences. The direct fusion of promoter to reporter sequences would therefore lead to suboptimal levels of transcription. A commonly used procedure to analyze expression patterns and levels is through determination of the ‘steady state’ level of protein accumulation in a cell. Commonly used candidates for the reporter gene, known to those skilled in the art are beta-glucuronidase (GUS), chloramphenicol acetyl transferase (CAT) and proteins with fluorescent properties, such as green fluorescent protein (GFP) from Aequora victoria. In principle, however, many more proteins are suitable for this purpose, provided the protein does not interfere with essential plant functions. For quantification and determination of localization a number of tools are suited. Detection systems can readily be created or are available which are based on, e.g., immunochemical, enzymatic, fluorescent detection and quantification. Protein levels can be determined in plant tissue extracts or in intact tissue using in situ analysis of protein expression. Generally, individual transformed lines with one chimeric promoter reporter construct may vary in their levels of expression of the reporter gene. Also frequently observed is the phenomenon that such transformants do not express any detectable product (RNA or protein). The variability in expression is commonly ascribed to ‘position effects’, although the molecular mechanisms underlying this inactivity are usually not clear.

The expression of the polynucleotide of interest can be determined by various well known techniques, e.g., by Northern Blot or in situ hybridization techniques as described in WO 02/102970.

### Nucleic Acids

The term “nucleic acid” as used herein refers to deoxyribonucleotides or ribonucleotides and their polymers thereof in either single- or double-stranded form, composed of monomers (nucleotides) containing a sugar, phosphate and a base, which is either a purine or pyrimidine. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer 1991; Ohtsuka 1985; Rossolini 1994). A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid” or “nucleic acid sequence” may also be used interchangeably with gene, cDNA, DNA and RNA encoded by a gene.

Isolated or substantially purified nucleic acid or protein compositions are also contemplated. The terms “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein of interest chemicals. The nucleotide sequences of the invention include both the naturally occurring sequences as well as mutant (variant) forms. Such variants will continue to possess the desired activity, i.e., either promoter activity or the activity of the product encoded by the open reading frame of the non-variant nucleotide sequence.

Nucleic acid variants of the transcription regulating nucleotide sequence that retain the activity of the wild-type transcription regulating nucleotide sequence are also contemplated. The term “variant” as used herein with respect to a sequence (e.g., a polypeptide or nucleic acid sequence such as—for example—a transcription regulating nucleotide sequence of the invention) is intended to mean substantially similar sequences. Naturally occurring allelic variants such as these can be identified with the use of well-known molecular biology techniques, as, for example, with polymerase chain reaction (PCR) and hybridization techniques.

Variant nucleotide sequences also include synthetically derived nucleotide sequences, such as those generated, for example, by using site-directed mutagenesis. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence set forth in SEQ ID NO: 1, SEQ ID NO: 2, SEQ ID NO: 3, SEQ ID NO: 4, SEQ ID NO: 5, or SEQ ID NO: 6 or a functional fragment thereof.

As used herein, the term “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

The term “substantial identity” of polynucleotide sequences means that a polynucleotide comprises a sequence that has at least 38%, e.g., 39%, 40%, 42%, 44%, 46%, 48%, 50%, 52%, 54%, 56%, 58%, 60%, 62%, 64%, 65%, 66%, 67%, 68%, 69%, 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like.

Another indication that nucleotide sequences are substantially identical is if two molecules hybridize to each other under stringent conditions (see below). Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

Stringent hybridization conditions” and “stringent hybridization wash conditions” in the context of nucleic acid hybridization experiments such as Southern and Northern hybridization are sequence dependent, and are different under different environmental parameters. The Tm is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tm can be approximated from the equation of Meinkoth and Wahl, 1984:

Tm=81.5° C.+16.6(log 10M)+0.41(% GC)−0.61(% form)−500/L
where M is the molarity of monovalent cations, % GC is the percentage of guanosine and cytosine nucleotides in the DNA, % form is the percentage of formamide in the hybridization solution, and L is the length of the hybrid in base pairs. Tm is reduced by about 1° C. for each 1% of mismatching; thus, Tm, hybridization, and/or wash conditions can be adjusted to hybridize to sequences of the desired identity. For example, if sequences with >90% identity are sought, the Tm can be decreased 10° C. Generally, stringent conditions are selected to be about 5° C. lower than the thermal melting point I for the specific sequence and its complement at a defined ionic strength and pH. However, severely stringent conditions can utilize a hybridization and/or wash at 1, 2, 3, or 4° C. lower than the thermal melting point I; moderately stringent conditions can utilize a hybridization and/or wash at 6, 7, 8, 9, or 10° C. lower than the thermal melting point I; low stringency conditions can utilize a hybridization and/or wash at 11, 12, 13, 14, 15, or 20° C. lower than the thermal melting point I. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tm for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4 to 6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tm for a particular probe. An example of highly stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

The following are examples of sets of hybridization/wash conditions that may be used to clone nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO4, 1 mM EDTA at 50° C. with washing in 2×SSC, 0.1% SDS at 50° C. (very low stringency conditions), more desirably in 7% sodium dodecyl sulfate (SDS), 0.5 M NaPO4, 1 mM EDTA at 50° C. with washing in 1×SSC, 0.1% SDS at 50° C. (low stringency conditions), more desirably still in 7% sodium dodecyl sulfate (SDS), 0.5 M NaPO4, 1 mM EDTA at 50° C. with washing in 0.5×SSC, 0.1% SDS at 50° C. (moderate stringency conditions), preferably in 7% sodium dodecyl sulfate (SDS), 0.5 M NaPO4, 1 mM EDTA at 50° C. with washing in 0.1×SSC, 0.1% SDS at 50° C. (high stringency conditions), more preferably in 7% sodium dodecyl sulfate (SDS), 0.5 M NaPO4, 1 mM EDTA at 50° C. with washing in 0.1×SSC, 0.1% SDS at 65° C. (very high stringency conditions).

In some embodiments, the nucleic acid molecules described herein can be “optimized” for enhanced expression in plants of interest (see, for example, WO 91/16432; Perlak 1991; Murray 1989). In this manner, the open reading frames in genes or gene fragments can be synthesized utilizing plant-preferred codons (see, for example, Campbell & Gowri, 1990 for a discussion of host-preferred codon usage). Thus, the nucleotide sequences can be optimized for expression in any plant. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art (see, for example, Stemmer 1994; Stemmer 1994; Crameri 1997; Moore 1997; Zhang 1997; Crameri 1998; and U.S. Pat. No. 5,605,794, 6, 8, 10, and 12,837,458).

### Polynucleotides of Interest

The term “polynucleotide of interest” as used herein refers to a nucleic acid which is expressed under the control of the transcription regulating nucleotide sequence referred to herein. Preferably, a polynucleotide of interest encodes a polypeptide the presence of which is desired in a plant cell, a plant, or a plant part as referred to herein. Such a polypeptide may be an enzyme which is required for the synthesis of seed storage compounds or may be a seed storage protein. It is to be understood that if the polynucleotide of interest encodes a polypeptide, transcription of the nucleic acid in RNA and translation of the transcribed RNA into the polypeptide may be required. A polynucleotide of interest, also preferably, includes biologically active RNA molecules and, more preferably, antisense RNAs, ribozymes, micro RNAs or siRNAs. For example, an undesired enzymatic activity in a seed can be reduced due to the seed specific expression of an antisense RNAs, ribozymes, micro RNAs or siRNAs. The underlying biological principles of action of the aforementioned biologically active RNA molecules are well known in the art. Moreover, the person skilled in the art is well aware of how to obtain nucleic acids which encode such biologically active RNA molecules. It is to be understood that the biologically active RNA molecules may be directly obtained by transcription of the nucleic acid of interest, i.e. without translation into a polypeptide. Preferably, at least one polynucleotide of interest to be expressed under the control of the transcription regulating nucleotide sequence of the present invention is heterologous in relation to said the transcription regulating nucleotide sequence, i.e. it is not naturally under the control thereof, but said control has been produced in a non-natural manner (for example by genetic engineering processes)

An operable linkage in relation to any expression cassette described herein may be realized by various methods known in the art, comprising both in vitro and in vivo procedure. Thus, an expression cassette of the invention or an vector comprising such expression cassette may by realized using standard recombination and cloning techniques well known in the art (see e.g., Maniatis 1989; Silhavy 1984; Ausubel 1987).

An operable linkage may—for example—comprise a sequential arrangement of the transcription regulating nucleotide sequence described herein (for example, the nucleotide sequence set forth in SEQ ID NO: 1, SEQ ID NO: 2, SEQ ID NO: 3, SEQ ID NO: 4, SEQ ID NO: 5, or SEQ ID NO: 6 or functional fragment thereof) with a nucleic acid sequence to be expressed, and—optionally—additional regulatory elements such as for example polyadenylation or transcription termination elements, enhancers, introns, etc., in a way that the transcription regulating nucleotide sequence can fulfill its function in the process of expressing the nucleic acid sequence of interest under the appropriate conditions. The term “appropriate conditions” mean preferably the presence of the expression cassette in a plant cell. Preferred are arrangements, in which the nucleic acid sequence of interest to be expressed is placed down-stream (i.e., in 3′-direction) of the transcription regulating nucleotide sequence of the invention in a way, that both sequences are covalently linked. Optionally additional sequences may be inserted in-between the two sequences. Such sequences may be for example linker or multiple cloning sites. Furthermore, sequences can be inserted coding for parts of fusion proteins (in case a fusion protein of the protein encoded by the nucleic acid of interest is intended to be expressed). Preferably, the distance between the polynucleotide of interest to be expressed and the transcription regulating nucleotide sequence of the invention is not more than 200 base pairs, preferably not more than 100 base pairs, more preferably no more than 50 base pairs.

In some embodiments, an expression cassette is assembled by inserting a transcription regulating nucleotide sequence described herein (for example a nucleotide sequence set forth in SEQ ID NO: 1, SEQ ID NO: 2, SEQ ID NO: 3, SEQ ID NO: 4, SEQ ID NO: 5, or SEQ ID NO: 6 or functional fragment thereof) into the plant genome. Such insertion will result in an operable linkage to a nucleic acid sequence of interest, which as such already existed in the genome. By the insertion, the nucleic acid of interest is expressed in a tissue-specific way due to the transcription regulating properties of the transcription regulating nucleotide sequence. The insertion may be directed or by chance. Preferably the insertion is directed and realized by for example homologous recombination. By this procedure a natural promoter may be exchanged against the transcription regulating nucleotide sequence of the invention, thereby modifying the expression profile of an endogenous gene. The transcription regulating nucleotide sequence may also be inserted in a way, that antisense mRNA of an endogenous gene is expressed, thereby inducing gene silencing.

Similar, a polynucleotide of interest to be expressed may by inserted into a plant genome comprising the transcription regulating nucleotide sequence in its natural genomic environment (i.e. linked to its natural gene) in a way that the inserted sequence becomes operably linked to the transcription regulating nucleotide sequence, thereby forming an expression cassette of the invention.

The expression cassette may be employed for numerous expression purposes such as for example expression of a protein, or expression of an antisense RNA, sense or double-stranded RNA. Preferably, expression of the nucleic acid sequence confers to the plant an agronomically valuable trait.

In some embodiments, the polynucleotide of interest is obtained from an insect resistance gene; a disease resistance gene such as, for example, a bacterial disease resistance gene, a fungal disease resistance gene, a viral disease resistance gene, or a nematode disease resistance gene; a herbicide resistance gene; a gene affecting grain composition or quality; a nutrient utilization gene; a mycotoxin reduction gene; a male sterility gene; a selectable marker gene; a screenable marker gene; a negative selectable marker; a positive selectable marker; a gene affecting plant agronomic characteristics, i.e., yield, standability, and the like; or an environment or stress resistance gene, i.e., one or more genes that confer herbicide resistance or tolerance, insect resistance or tolerance, disease resistance or tolerance (viral, bacterial, fungal, oomycete, or nematode), stress tolerance or resistance (as exemplified by resistance or tolerance to drought, heat, chilling, freezing, excessive moisture, salt stress, or oxidative stress), increased yields, food content and makeup, physical appearance, male sterility, dry down, standability, prolificacy, starch properties or quantity, oil quantity and quality, amino acid or protein composition, and the like.

By “resistant” is meant a plant, which exhibits substantially no phenotypic changes as a consequence of agent administration, infection with a pathogen, or exposure to stress. By “tolerant” is meant a plant, which, although it may exhibit some phenotypic changes as a consequence of infection, does not have a substantially decreased reproductive capacity or substantially altered metabolism.

In some embodiments, the polynucleotide of interest is a selectable marker gene. The term “selectable marker gene” as used herein, refers to a gene that—in the presence of the corresponding selection compound (e.g., herbicide) in the growing medium—confers a growth advantage to a plant or plant cell transformed with a plant expression cassette for said selectable marker as compared to a plant or plant cell not been transformed with said plant expression cassette and which, thus, does not comprise the selectable marker gene. Preferably, the selectable marker gene and/or plant expression cassette for said marker gene is heterologous to the plant to be transformed, and thus is not naturally present in the plant to be transformed.

In some embodiments, the selectable marker gene is a negative selection marker gene. Negative selection marker genes confer a resistance and/or increased tolerance to a selection compound (e.g., herbicide). Exemplary selectable marker genes include, but are not limited to, Phosphinothricin acetyltransferases (PAT; also named Bialaphoeresistance; bar; De Block et al. (1987) Plant Physiol 91:694-701; EP 0 333 033; U.S. Pat. No. 4,975,374) 5-enolpyruvylshikimate-3-phosphate synthase (EPSPS; U.S. Pat. No. 5,633,435) or glyphosate oxidoreductase gene (U.S. Pat. No. 5,463,175) conferring resistance to Glyphosate™ (N-(phosphonomethyl)glycine) (Shah of al. (1986) Science 233:478) Glyphosate. TM. degrading enzymes (Glyphosate™ oxidoreductase; gox), Sulfonylurea- and imidazolinone-inactivating acetolactate synthases (for example mutated ALS variants with, for example, the S4 and/or Hra mutation Bromoxynil™ degrading nitrilases (bxn) Kanamycin- or. G418-resistance genes (NPTII; NPTI) coding e.g., for neomycin phosphotransferases (Fraley et al. (1983) Proc Natl Acad Sci USA 80:4803), which expresses an enzyme conferring resistance to the antibiotic kanamycin and the related antibiotics neomycin, paromomycin, gentamicin, and G418, Dicamba degrading enzymes (O-demethylase, oxygenase, ferredoxin) (Behrens et al. 2007 Science 316:1185-1188; U.S. Pat. No. 7,022,896) marker genes that confer resistance against the toxic effects imposed by D-amino acids like e.g., D-alanine and D-serine (WO03/060133). Especially preferred as marker genes in this contest are the daol gene (EC: 1.4. 3.3: GenBank Acc.-No.: U60066) from the yeast Rhodotorula gracilis (Rhodosporidium toruloides) and the E. coli gene dsdA (D-serine dehydratase (D-serine deaminase) [EC: 4.3. 1.18; GenBank Acc.-No.: J01603).

In some embodiments, the selectable marker gene is a positive selection marker, which confers a growth advantage to a transformed plant in comparison with a non-transformed one. Exemplary positive selection markers include, but are not limited to, mannose-6-phosphate isomerase (in combination with mannose), UDPgalactose-4-epimerase (in combination with e.g., galactose), wherein mannose-6-phosphate isomerase in combination with mannose is especially preferred.

In some embodiments, the selectable marker gene is the acetohydroxy acid synthase (AHAS) gene, or a mutated AHAS gene. The acetohydroxy acid synthase enzyme (also known as acetolactate synthase, or ALS) is a protein found in plants and microorganisms and which catalyzes the first step in the synthesis of the branched-chain amino acids (valine, leucine, and isoleucine). Preferably, it has enzymatic activity as set forth in the Enzyme Commission Code EC 2.2.1.6. The mutated AHAS protein, preferably, confers resistance to at least one imidazolinone herbicide. Imidazolinone herbicides are well known in the art, and, preferably, include imazapyr, imazaquin, imazethapyr, imazapic, imazamox and imazamethabenz. Preferably, the imidazolinone herbicide is imazaquin. More preferably, the imidazolinone herbicide is imazethapyr. Most preferably, the imidazolinone herbicide is imazapyr.

Exemplary mutated AHAS genes are disclosed in WO2004/005516 or WO2008/124495 which herewith is incorporated by reference with respect to its entire disclosure content. Further preferred mutated AHAS genes are disclosed in WO2006/015376 or WO2007/054555 or US20100287641. The mutated AHAS enzyme confers resistance to imidazolinone herbicides.

Further selection marker genes are marker genes that confer resistance or increased tolerance to the toxic effects imposed by D-amino acids. Such preferred marker genes, preferably, encode for proteins which are capable of metabolizing D-amino acids. Preferred D-amino acids are D-alanine and D-serine. Particularly preferred marker genes encode for D-serine ammonialyases, D-amino acid oxidases and D-alanine transaminases. Preferred examples for such marker genes encoding for proteins which are capable of metabolizing D-amino acids are those which are as disclosed in International Patent Publication Nos. WO 03/060133, WO 05/090584, WO 07/107,516 and WO 08/077,570 which are incorporated herein by reference in their entirety.

In some embodiments, the polynucleotide of interest in a herbicide resistant gene encoding a herbicide resistant protein. Exemplary herbicide resistant genes include, but are not limited to the genes encoding phosphinothricin acetyltransferase (bar and pat), glyphosate tolerant EPSP synthase genes, the glyphosate degradative enzyme gene gox encoding glyphosate oxidoreductase, deh (encoding a dehalogenase enzyme that inactivates dalapon), herbicide resistant (e.g., sulfonylurea and imidazolinone) acetolactate synthase, and bxn genes (encoding a nitrilase enzyme that degrades bromoxynil). The bar and pat genes code for an enzyme, phosphinothricin acetyltransferase (PAT), which inactivates the herbicide phosphinothricin and prevents this compound from inhibiting glutamine synthetase enzymes. The enzyme 5-enolpyruvylshikimate 3-phosphate synthase (EPSP Synthase), is normally inhibited by the herbicide N-(phosphonomethyl)glycine (glyphosate). However, genes are known that encode glyphosate-resistant EPSP Synthase enzymes. The deh gene encodes the enzyme dalapon dehalogenase and confers resistance to the herbicide dalapon. The bxn gene codes for a specific nitrilase enzyme that converts bromoxynil to a non-herbicidal degradation product.

In some embodiments, the polynucleotide of interest is an insect resistant gene or a variant thereof encoding an insect resistant protein. Such variants can include synthetically derived sequences including but not limited to sequences that are a fusion of two or more polynucleotides of interest (e.g., two or more insect resistant genes). Exemplary insect resistant genes include, but are not limited to, genes that encode insecticidal proteins such as the Cry and Cyt proteins as well as genes that encode insecticidal proteins such as the “Vip” proteins. Examples of such genes include Cry1, such as members of the Cry1A, Cry1B, Cry1C, Cry1D, Cry1E, Cry1F, and Cr1I families; Cry2, such as members of the Cry2A family; Cry9, such as members of the Cry9A, Cry9B, Cry9C, Cry9D, Cry9E, and Cry9F families; and members of the Vip3 family, etc. It will be understood by one of skill in the art that the transgenic plant may comprise any gene imparting an agronomic trait of interest. Exemplary insect resistant genes include, but are not limited to, Bacillus thuringiensis crystal toxin genes or Bt genes (Watrud 1985). Bt genes may provide resistance to lepidopteran or coleopteran pests such as European Corn Borer (ECB) and corn rootworm (CRW). Preferred Bt toxin genes for use in such embodiments include the CryIA (b) and CryIA (c) genes. Endotoxin genes from other species of B. thuringiensis, which affect insect growth or development, may also be employed in this regard. Protease inhibitors may also provide insect resistance (Johnson 1989), and will thus have utility in plant transformation. The use of a protease inhibitor II gene, pinII, from tomato or potato is envisioned to be particularly useful. Other genes, which encode inhibitors of the insects' digestive system, or those that encode enzymes or co-factors that facilitate the production of inhibitors, may also be useful. Cystatin and amylase inhibitors, such as those from wheat and barley, may exemplify this group.

Also, genes encoding lectins may confer additional or alternative insecticide properties. Lectins (originally termed phytohemagglutinins) are multivalent carbohydrate-binding proteins, which have the ability to agglutinate red blood cells from a range of species. Lectins have been identified recently as insecticidal agents with activity against weevils, ECB and rootworm (Murdock 1990; Czapla & Lang, 1990). Lectin genes contemplated to be useful include, for example, barley and wheat germ agglutinin (WGA) and rice lectins (Gatehouse 1984), with WGA being preferred.

Genes controlling the production of large or small polypeptides active against insects when introduced into the insect pests, such as, e.g., lytic peptides, peptide hormones and toxins and venoms, form another aspect of the invention. For example, it is contemplated, that the expression of juvenile hormone esterase, directed towards specific insect pests, may also result in insecticidal activity, or perhaps cause cessation of metamorphosis (Hammock 1990).

### Transgenic Plants and Host Cells

Transgenic host cells or non-human, transgenic organisms comprising an expression cassette described herein are also contemplated. Preferred are prokaryotic and eukaryotic organisms. Both microorganism and higher organisms are comprised. Preferred microorganisms are bacteria, yeast, algae, and fungi. Preferred bacteria are those of the genus Escherichia, Erwinia, Agrobacterium, Flavobacterium, Alcaligenes, Pseudomonas, Bacillus or Cyanobacterim such as—for example—Synechocystis and other bacteria described in Brock Biology of Microorganisms Eighth Edition (pages A-8, A-9, A10 and A11). In some embodiments, the transgenic cells or non-human, transgenic organisms comprise an expression cassette described herein is a plant cell or plant (as defined herein). In some embodiments, the plant is hemizygous for the expression cassette. In some embodiments, the plant is homozygous for the expression cassette.

Especially preferred are microorganisms capable to infect plants and to transfer DNA into their genome, especially bacteria of the genus Agrobacterium, preferably Agrobacterium tumefaciens and rhizogenes. Preferred yeasts are Candida, Saccharomyces, Hansenula and Pichia. Preferred fungi are Aspergillus, Trichoderma, Ashbya, Neurospora, Fusarium, and Beauveria.

In some embodiments, the host cell is a plant cell, plant, a plant seed, a non-human animal or a multicellular microorganism. The term “plant” as used herein refers to a photosynthetic, eukaryotic multicellular organism. Plants encompass green algae (Chlorophyta), red algae (Rhodophyta), Glaucophyta, mosses and liverworts (bryophytes), seedless vascular plants (horsetails, club mosses, ferns) and seed plants (angiosperms and gymnosperms). The term “plant” encompasses whole plants, ancestors and progeny of the plants and plant parts, including seeds, shoots, stems, leaves, roots, flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen, microspores and propagules, again wherein each of the aforementioned comprises the gene/nucleic acid of interest.

The term “plant parts” as used herein encompasses seeds, shoots, stems, leaves, roots, flowers, and tissues and organs, plant cells, suspension cultures, callus tissue, embryos, meristematic regions, gametophytes, sporophytes, pollen, microspores and propagules. A “Propagule” is any kind of organ, tissue, or cell of a plant capable of developing into a complete plant. A propagule can be based on vegetative reproduction (also known as vegetative propagation, vegetative multiplication, or vegetative cloning) or sexual reproduction. A propagule can therefore be seeds or parts of the non-reproductive organs, like stem or leave. In particular, with respect to Poaceae, suitable propagules can also be sections of the stem, i.e., stem cuttings.

A transgenic plant cell, plant tissue, plant organ, or plant seed, comprising an expression cassette or a vector described herein is specifically contemplated. The expression cassette or vector may be present in the cytoplasm of the organism or may be incorporated into the genome either heterologous or by homologous recombination. Host cells, in particular those obtained from plants or animals, may be introduced into a developing embryo in order to obtain mosaic or chimeric organisms, i.e. transgenic organisms, i.e. plants, comprising the host cells of described herein. Suitable transgenic organisms are, preferably, all organisms which are suitable for the expression of recombinant genes.

Transgenic plants expressing genes, which encode enzymes that affect the integrity of the insect cuticle form yet another aspect of the invention. Such genes include those encoding, e.g., chitinase, proteases, lipases and also genes for the production of nikkomycin, a compound that inhibits chitin synthesis, the introduction of any of which is contemplated to produce insect resistant maize plants. Genes that code for activities that affect insect molting, such those affecting the production of ecdysteroid UDP-glucosyl transferase, also fall within the scope of the useful transgenes of the present invention.

Genes that code for enzymes that facilitate the production of compounds that reduce the nutritional quality of the host plant to insect pests are also encompassed by the present invention. It may be possible, for instance, to confer insecticidal activity on a plant by altering its sterol composition. Sterols are obtained by insects from their diet and are used for hormone synthesis and membrane stability. Therefore alterations in plant sterol composition by expression of novel genes, e.g., those that directly promote the production of undesirable sterols or those that convert desirable sterols into undesirable forms, could have a negative effect on insect growth and/or development and hence endow the plant with insecticidal activity. Lipoxygenases are naturally occurring plant enzymes that have been shown to exhibit anti-nutritional effects on insects and to reduce the nutritional quality of their diet. Therefore, further embodiments of the invention concern transgenic plants with enhanced lipoxygenase activity which may be resistant to insect feeding.

The nature of the transgenic plant cells, plants, and plant parts are not limited; for example, the plant cell can be monocotyledonous or dicotyledonous. In some embodiments, the transgenic plant transgenic plant tissue, plant organ, plant or seed is a monocotyledonous plant or a plant cell, plant tissue, plant organ, plant seed from a monocotyledonous plant. In some embodiments, the transgenic plant transgenic plant tissue, plant organ, plant or seed is a monocotyledonous plant or a plant cell, plant tissue, plant organ, plant seed from a dicotyledonous plant. Examples of transgenic plant cells finding use according to the disclosure include, but are not limited to, cells (or entire plants or plant parts) derived from the genera: Ananas, Musa, Vitis, Fragaria, Lotus, Medicago, Onobrychis, Trifolium, Trigonella, Vigna, Citrus, Carica, Persea, Prunus, Syragrus, Theobroma, Coffea, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Mangifera, Cichorium, Helianthus, Lactuca, Bromus, Asparagus, Antirrhinum, Heterocallis, Nemesia, Pelargonium, Panicum, Pennisetum, Ranunculus, Senecio, Salpiglossis, Cucurbita, Cucumis, Browaalia, Lolium, Malus, Apium, Gossypium, Vicia, Lathyrus, Lupinus, Pachyrhizus, Wisteria, Stizolobium, Agrostis, Phleum, Dactylis, Sorghum, Setaria, Zea, Oryza, Triticum, Secale, Avena, Hordeum, Saccharum, Poa, Festuca, Stenotaphrum, Cynodon, Coix, Olyreae, Phareae, Glycine, Pisum, Psidium, Passiflora, Cicer, Phaseolus, Lens, and Arachis.

In some embodiments the transgenic plant cells include cells (or entire plants or plant parts) from the family of poaceae, such as the genera Hordeum, Secale, Avena, Sorghum, Andropogon, Holcus, Panicum, Oryza, Zea, Triticum, for example the genera and species Hordeum vulgare, Hordeum jubatum, Hordeum murinum, Hordeum secalinum, Hordeum distichon, Hordeum aegiceras, Hordeum hexastichon, Hordeum hexastichum, Hordeum irregulare, Hordeum sativum, Hordeum secalinum, Secale cereale, Avena sativa, Avena fatua, Avena byzantina, Avena fatua var. sativa, Avena hybrida, Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cermum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum, Panicum militaceum, Oryza sativa, Oryza latifolia, Zea mays, Triticum aestivum, Triticum durum, Triticum turgidum, Triticum hybernum, Triticum macha, Triticum sativum or Triticum vulgare.

In some embodiments, plants to be used as transgenic plants are oil fruit crops which comprise large amounts of lipid compounds, such as peanut, oilseed rape, canola, sunflower, safflower, poppy, mustard, hemp, castor-oil plant, olive, sesame, Calendula, Punica, evening primrose, mullein, thistle, wild roses, hazelnut, almond, macadamia, avocado, bay, pumpkin/squash, linseed, soybean, pistachios, borage, trees (oil palm, coconut, walnut) or crops such as maize, wheat, rye, oats, triticale, rice, barley, cotton, cassava, pepper, Tagetes, Solanaceae plants such as potato, tobacco, eggplant and tomato, Vicia species, pea, alfalfa or bushy plants (coffee, cacao, tea), Salix species, and perennial grasses and fodder crops. Preferred plants according to the invention are oil crop plants such as peanut, oilseed rape, canola, sunflower, safflower, poppy, mustard, hemp, castor-oil plant, olive, Calendula, Punica, evening primrose, pumpkin/squash, linseed, soybean, borage, trees (oil palm, coconut).

Methods for producing transgenic tissue, plant organ, plant or seed comprising introducing an expression cassette of vector described herein into a plant cell and regenerating the plant cell to form a plant tissue, plant organ, plant or seed are also contemplated.

Methods of providing pesticidal activity to a plant comprising introducing an expression cassette of vector described herein comprising a nucleotide sequences that encodes a pesticidal protein into a plant cell and regenerating the plant cell to form a plant tissue, plant organ, plant or seed, thereby providing pesticidal activity to the plant, are also contemplated. In some embodiments, the pesticidal activity is insecticidal activity.

Expression cassettes can be introduced into plant cells in a number of art-recognized ways. Plant species may be transformed with the DNA construct described herein by the DNA-mediated transformation of plant cell protoplasts and subsequent regeneration of the plant from the transformed protoplasts in accordance with procedures well known in the art.

Any plant tissue capable of subsequent clonal propagation, whether by organogenesis or embryogenesis, may be transformed with a vector described herein. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and ultilane meristem).

Plants may take a variety of forms. For example, the plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npt II) can be associated with the expression cassette to assist in breeding.

Transformation of plants can be undertaken with a single DNA molecule or multiple DNA molecules (i.e., co-transformation), and both these techniques are suitable for use with the expression cassettes described herein. Numerous transformation vectors are available for plant transformation, and the expression cassettes of this invention can be used in conjunction with any such vectors. The selection of vector will depend upon the preferred transformation technique and the target species for transformation.

A variety of techniques are available and known to those skilled in the art for introduction of constructs into a plant cell host. Exemplary techniques include transformation with DNA employing A. tumefaciens or A. rhizogenes as the transforming agent, liposomes, PEG precipitation, electroporation, DNA injection, direct DNA uptake, microprojectile bombardment, particle acceleration, and the like (See, for example, EP 295959 and EP 138341) (see below). However, cells other than plant cells may be transformed with the expression cassettes described herein. The general descriptions of plant expression vectors and reporter genes, and Agrobacterium and Agrobacterium-mediated gene transfer, can be found in Gruber et al. (1993).

Expression vectors containing genomic or synthetic fragments can be introduced into protoplasts or into intact tissues or isolated cells. Preferably expression vectors are introduced into intact tissue. General methods of culturing plant tissues are provided for example by Maki et al., (1993); and by Phillips et al. (1988). Preferably, expression vectors are introduced into maize or other plant tissues using a direct gene transfer method such as microprojectile-mediated delivery, DNA injection, electroporation and the like. More preferably expression vectors are introduced into plant tissues using the microprojectile media delivery with the biolistic device. See, for example, Tomes et al. (1995). The vectors of the invention can not only be used for expression of structural genes but may also be used in exon-trap cloning, or promoter trap procedures to detect differential gene expression in varieties of tissues (Lindsey 1993; Auch & Reth 1990).

In some embodiments, the binary type vectors of Ti and Ri plasmids of Agrobacterium spp. Ti-derived vectors are used to transform a wide variety of higher plants, including monocotyledonous and dicotyledonous plants, such as soybean, cotton, rape, tobacco, and rice (Pacciotti 1985: Byrne 1987; Sukhapinda 1987; Lorz 1985; Potrykus, 1985; Park 1985: Hiei 1994). The use of T-DNA to transform plant cells has received extensive study and is amply described (EP 120516; Hoekema, 1985; Knauf, 1983; and An 1985).

Other transformation methods are available to those skilled in the art, such as direct uptake of foreign DNA constructs (see EP 295959), techniques of electroporation (Fromm 1986) or high velocity ballistic bombardment with metal particles coated with the nucleic acid constructs (Kline 1987, and U.S. Pat. No. 4,945,050). Once transformed, the cells can be regenerated by those skilled in the art. Of particular relevance are the recently described methods to transform foreign genes into commercially important crops, such as rapeseed (De Block 1989), sunflower (Everett 1987), soybean (McCabe 1988; Hinchee 1988; Chee 1989; Christou 1989; EP 301749), rice (Hiei 1994), and corn (Gordon-Kamm 1990; Fromm 1990).

Those skilled in the art will appreciate that the choice of method might depend on the type of plant, i.e., monocotyledonous or dicotyledonous, targeted for transformation. Suitable methods of transforming plant cells include, but are not limited to, microinjection (Crossway 1986), electroporation (Riggs 1986), Agrobacterium-mediated transformation (Hinchee 1988), direct gene transfer (Paszkowski 1984), and ballistic particle acceleration using devices available from Agracetus, Inc., Madison, Wis. And BioRad, Hercules, Calif. (see, for example, U.S. Pat. No. 4,945,050; and McCabe 1988). Also see, Weissinger 1988; Sanford 1987 (onion); Christou 1988 (soybean); McCabe 1988 (soybean); Datta 1990 (rice); Klein 1988 (maize); Klein 1988 (maize); Klein 1988 (maize); Fromm 1990 (maize); and Gordon-Kamm 1990 (maize); Svab 1990 (tobacco chloroplast); Koziel 1993 (maize); Shimamoto 1989 (rice); Christou 1991 (rice); European Patent Application EP 0 332 581 (orchardgrass and other Pooideae); Vasil 1993 (wheat); Weeks 1993 (wheat).

Methods using either a form of direct gene transfer or Agrobacterium-mediated transfer usually, but not necessarily, are undertaken with a selectable marker, which may provide resistance to an antibiotic (e.g., kanamycin, hygromycin or methotrexate) or a herbicide (e.g., phosphinothricin). For certain plant species, different antibiotic or herbicide selection markers may be preferred. Selection markers used routinely in transformation include the nptII gene which confers resistance to kanamycin and related antibiotics (Messing & Vierra, 1982; Bevan 1983), the bar gene which confers resistance to the herbicide phosphinothricin (White 1990, Spencer 1990), the hph gene which confers resistance to the antibiotic hygromycin (Blochlinger & Diggelmann), and the dhfr gene, which confers resistance to methotrexate (Bourouis 1983).

Methods for the production and further characterization of stably transformed plants are well-known to the person skilled in the art. As an example, transgenic plant cells are placed in an appropriate selective medium for selection of transgenic cells, which are then grown to callus. Shoots are grown from callus. Plantlets are generated from the shoot by growing in rooting medium. The various constructs normally will be joined to a marker for selection in plant cells. Conveniently, the marker may be resistance to a biocide (particularly an antibiotic, such as kanamycin, G418, bleomycin, hygromycin, chloramphenicol, herbicide, or the like). The particular marker used will allow for selection of transformed cells as compared to cells lacking the DNA, which has been introduced. Components of DNA constructs including transcription cassettes of this invention may be prepared from sequences, which are native (endogenous) or foreign (exogenous) to the host. By “foreign” it is meant that the sequence is not found in the wild-type host into which the construct is introduced. Heterologous constructs will contain at least one region, which is not native to the gene from which the transcription-initiation-region is derived.

To confirm the presence of the transferred polynucleotide of interest in transgenic cells and plants, a variety of assays may be performed. Such assays include, for example, “molecular biological” assays well known to those of skill in the art, such as Southern and Northern blotting, in situ hybridization and nucleic acid-based amplification methods such as PCR or RT-PCR or TaqMan; “biochemical” assays, such as detecting the presence of a protein product, e.g., by immunological means (ELISAs and Western blots) or by enzymatic function; plant part assays, such as seed assays; and also, by analyzing the phenotype of the whole regenerated plant, e.g., for disease or pest resistance.

DNA may be isolated from cell lines or any plant parts to determine the presence of the preselected nucleic acid segment through the use of techniques well known to those skilled in the art. Note that intact sequences will not always be present, presumably due to rearrangement or deletion of sequences in the cell.

In some embodiments, the presence of nucleic acid elements introduced through the methods of this invention may be determined by polymerase chain reaction (PCR). Using these technique discreet fragments of nucleic acid are amplified and detected by gel electrophoresis. This type of analysis permits one to determine whether a preselected nucleic acid segment is present in a stable transformant, but does not prove integration of the introduced preselected nucleic acid segment into the host cell genome. In addition, it is not possible using PCR techniques to determine whether transformants have exogenous genes introduced into different sites in the, genome, i.e., whether transformants are of independent origin. It is contemplated that using PCR techniques it would be possible to clone fragments of the host genomic DNA adjacent to an introduced preselected DNA segment.

Known methods of PCR include, but are not limited to, methods using paired primers, nested primers, single specific primers, degenerate primers, gene-specific primers, vector-specific primers, partially mismatched primers, and the like.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced preselected DNA segments in high molecular weight DNA, i.e., confirm that the introduced preselected, DNA segment has been integrated into the host cell genome. The technique of Southern hybridization provides information that is obtained using PCR, e.g., the presence of a preselected DNA segment, but also demonstrates integration into the genome and characterizes each individual transformant.

It is contemplated that using the techniques of dot or slot blot hybridization which are modifications of Southern hybridization techniques one could obtain the same information that is derived from PCR, e.g., the presence of a preselected DNA segment.

Both PCR and Southern hybridization techniques can be used to demonstrate transmission of a preselected DNA segment to progeny. In most instances the characteristic Southern hybridization pattern for a given transformant will segregate in progeny as one or more Mendelian genes (Spencer 1992); Laursen 1994) indicating stable inheritance of the gene. The non-chimeric nature of the callus and the parental transformants (RO) was suggested by germline transmission and the identical Southern blot hybridization patterns and intensities of the transforming DNA in callus, RO plants and RI progeny that segregated for the transformed gene.

Whereas DNA analysis techniques may be conducted using DNA isolated from any part of a plant, RNA may only be expressed in particular cells or tissue types and hence it will be necessary to prepare RNA for analysis from these tissues. PCR techniques may also be used for detection and quantitation of RNA produced from introduced preselected DNA segments. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and will only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the protein products of the introduced preselected DNA segments or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed.

Very frequently the expression of a gene product is determined by evaluating the phenotypic results of its expression. These assays also may take many forms including but not limited to analyzing changes in the chemical composition, morphology, or physiological properties of the plant. Morphological changes may include greater stature or thicker stalks. Most often changes in response of plants or plant parts to imposed treatments are evaluated under carefully controlled conditions termed bioassays.

It is to be understood that this invention is not limited to the particular methodology, proto-cols, cell lines, plant species or genera, constructs, and reagents described as such. It is also to be understood that the terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth.

### EXAMPLES

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Example 1: Identification and Analysis of Novel Promoters

### Introduction

A diverse genetic toolbox to drive transgene expression is important for gene discovery and trait optimization efforts. Diverse genetic elements can serve to increase transformation efficacy of genes, optimize transgene expression levels, and reduce silencing of transgenic constructs. Genetic elements are also important targets for genome editing purposes, as they can be modified, swapped, or truncated to alter gene expression. This includes cis-regulatory motifs present in promoters and other regions of the genome.

One objective of the study was to identify promoter sequences that showed high constitutive expression with low variation across different plant tissue types and developmental stages. To do this, Natural Language Processing (herein, “NLP”) methods (referenced in U.S. patent application Ser. No. 17/088,734-titled “APPARATUSES, SYSTEMS, AND METHODS FOR EXTRACTING MEANING FROM DNA SEQUENCE DATA USING NATURAL LANGUAGE PROCESSING (NLP)” and herein incorporated in its entirety by reference) were used to identify constitutively expressed promoters in soybean, as demonstrated by example in SEQ ID NOs: 2, 3 and 5, from Applicant proprietary soybean RNA-seq expression datasets. Applicants focused on identifying k-mers (DNA motifs) that were associated with high constitutive expression (transcript abundance) across multiple tissues, developmental stages, and independent experiments. Transcripts with high coefficients of variation were discarded from the dataset and only those transcripts with upstream DNA sequence were retained. These transcripts were binned by relative expression level across all experimental datasets as low (bottom 20%), medium (middle 30-60%), or high (top 20%).

Using this approach, the k-mer GATCTG was found to be associated with the high gene expression bin. The gene Glyma. 14G124300 (annotated as a ubiquitin conjugating enzyme; UBC and with annotated promoter sequence depicted in SEQ ID NO: 2, also referenced as UBC-n) contains three repeats of GATCTG near (e.g. at or less than about 2000 bp) the coding sequence (CDS) start site. A mutant form of this promoter was generated by deleting the three occurrences of GATCTG (as depicted in SEQ ID NO: 1, also referenced herein as UBC-m).

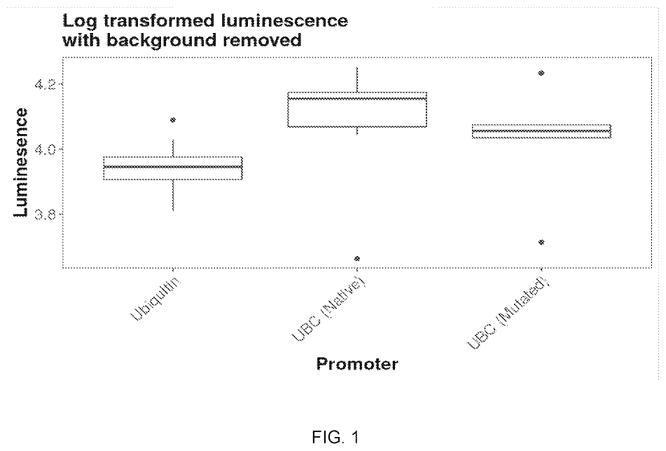

Applicants experimentally validated high expression levels of these promoter sequences using a luciferase assay in a transient tobacco system. High levels of expression of these two promoters were confirmed as compared to a positive control (Arabidopsis UBQ10; AT4G05320) as well as a slight decrease in expression of the mutant version of the promoter. It was surprisingly observed that deleting the occurrences of GATCTG resulted in a promoter that showed significantly less variance in expression compared to the native version, UBC-n (see FIG. 1).

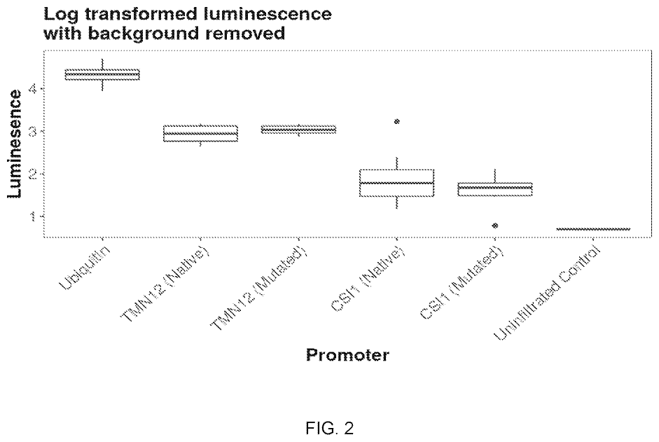

To validate the role of the k-mer GATCTG in reducing expression variation, two additional promoters containing copies of GATCTG were selected, as they were also identified using the described NLP method. The upstream region of Glyma. 13G026100 (depicted in SEQ ID NO: 3) contains three repeats of GATCTG at positions −220, −248, and −276 upstream of the CDS start site. The upstream region of Glyma. 18G019600 (depicted in SEQ ID NO: 5) contains four repeats of GATCTG at positions −96, −125, −188, and −355 upstream of the CDS start site. The upstream regions of Glyma.13G026100 and Glyma. 18G019600, with and without (those without, respectively depicted in SEQ ID NOs: 4 and 6), the GATCTG k-mer were experimentally tested using a luciferase assay in a transient tobacco system. For both promoters, it was observed that deleting the occurrences of GATCTG resulted in a promoter that showed significantly less variance in expression compared to the native version (see FIG. 2).

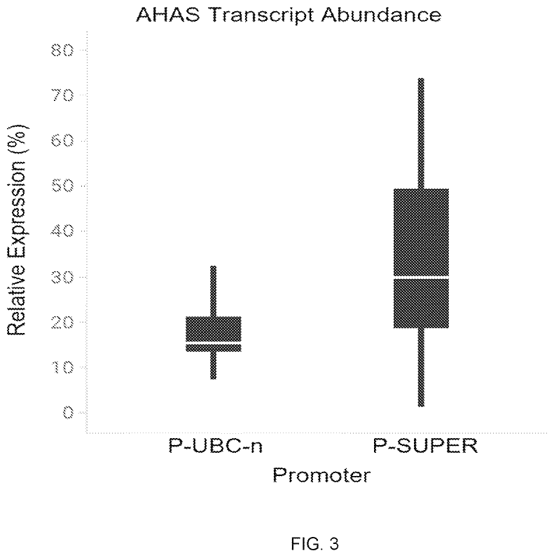

Validation of the transient expression data in the tobacco system for the native SEQ ID NO: 2, also referenced as UBC-n, was conducted by stable transformation in soybean. The UBC-n sequence was used to drive expression of AHAS. It was observed that UBC-n promoted expression of AHAS in stably transformed soybean. This stable expression was compared to a positive control, the SUPER promoter, as depicted in SEQ ID NO: 66 of U.S. Patent Publication 2013-0091598 (herein, “Super promoter”) (see FIG. 3).

### Methods

### K-Mer and Promoter Identification

NLP methods were used for k-mer and promoter discovery. Three RNA-seq datasets from soybean were used to mine expression data across tissue types and developmental stages.

### Luciferase Assay

DNA constructs having a luciferase expression cassette driven by different promoters (including Ubi-n and Ubi-m) were prepared using standard vector construction methods and introduced into Agrobacterium EHA105 strain via electroporation.

Prior to infiltration, Agrobacteria carrying different constructs were grown on YEP plates with proper selection for 24 hours. Bacteria were harvested and suspended in infiltration medium to make 0.5 OD₆₀₀ bacterial suspension. Multiple individual leaves from different plants (one leaf per plant) at the same growth stage (4 to 5-weeks old) were infiltrated with EHA105 suspension. The infiltration process was monitored visually by observing the spread of opacity in leaf tissue as the bacterial suspension fills leaf airspaces. Infiltrated areas were outlined with a marker and plants allowed to continue growth under artificial illumination.

Two days after infiltration, two leaf discs were sampled from each infiltrated leaf. Luciferase protein was extracted from leaf discs in 150 μL of 1×PBS using genome grinder. Cell debris were removed by centrifugation and the supernatant was frozen and stored at −80° C., until use in an in-vitro luciferase activity assay.

Quantitative measurement of luciferase activities was carried out using “Steady-Glo Luciferase Assay System” (Promega catalog number PR-E2520) in a 96 well plate (Fisher Scientific catalog number 07-200-589) per Promega instruction. Protein luciferase level in each sample was then calculated based on luciferase activity detected and the standard curve of recombinant luciferase purchased from Promega (catalog number PR-E1701).

### Results

The promoter from Glyma. 14G124300 (UBC-n or UBC-native) was identified as being associated with highly expressed genes across multiple tissue types and developmental stages. Additionally, this gene contained three repeats of the k-mer GATCTG near the CDS start site that was identified in multiple models using NLP methods as being associated with transcripts in the high constitutive expression bin. A mutant version of the promoter was generated by deleting the GATCTG motifs at −23, −46, and −69 bp upstream of the CDS start site (UBC-m or UBC-mutated, depicted in SEQ ID NO: 1).

To validate the expression level of the UBC promoter, we used the promoter to drive expression of luciferase in a transient tobacco system. We used 1 kb upstream of the CDS start site of UBC as the promoter. As a positive control, we used 1307 bp of the promoter plus the first intron of Ubiquitin (UBQ10) from Arabidopsis and an uninfiltrated leaf as a negative control. We observed that both the native and mutant promoter of UBC resulted in higher expression compared to the positive UBQ10 control (FIG. 1). Moreover, we observed less expression variation for the mutant promoter that lacked the three GATCTG k-mers near the CDS start site.

To validate the role of the k-mer GATCTG in reducing expression variation, two additional promoters containing copies of GATCTG were selected. The upstream regions of Glyma. 13G026100 and Glyma. 18G019600, with and without GATCTG k-mers were tested. For both promoters, it was observed that deletion of the GATCTG k-mer reduced expression variation in the transient expression system

Validation of the transient expression data for the native SEQ ID NO: 2, also referenced as UBC-n, by stable transformation demonstrated that UBC-n can drive expression of a transgene (AHAS) in soybean.

### CONCLUSION

Applicants identified the promoter from Glyma. 14G124300 (UBC) that resulted in high constitutive expression of luciferase in a transient tobacco system compared to the positive control (UBQ10 from Arabidopsis). Testing used the native sequence as well as a mutant form of the UBC promoter by deleting three repeats of GATCTG near the CDS start site. Unexpectedly, deletion of GATCTG resulted in less variation in expression with the mutant version. Both the native and mutant forms of the UBC promoter will be valuable for driving transgene expression in planta. The reduction in expression variation from GATCTG disruption was validated by deleting copies of GATCTG from the promoters of two additional genes, Glyma. 13G026100 (TMN12) and Glyma.18G019600 (CSI12). Both native and mutant promoters drove reporter gene expression and as seen with the UBC promoter, deletion of GATCTG decreased variation in expression. The ability of the Glyma. 14G124300 (UBC) promoter to drive gene expression was further validated by using stable transformation of soybean.

### Example 2. Soybean Transformation

Soybean transformation is achieved using methods well known in the art, such as the one described using the Agrobacterium tumefaciens mediated transformation soybean half-seed explants using essentially the method described by Paz et al. (2006), Plant cell Rep. 25:206. Transformants are identified using tembotrione as selection marker. The appearance of green shoots was observed, and documented as an indicator of tolerance to the herbicide isoxaflutole or tembotrione. The tolerant transgenic shoots will show normal greening comparable to wild-type soybean shoots not treated with isoxaflutole or tembotrione, whereas wild-type soybean shoots treated with the same amount of isoxaflutole or tembotrione will be entirely bleached. This indicates that the presence of the HPPD protein enables the tolerance to HPPD inhibitor herbicides, like isoxaflutole or tembotrione.

Tolerant green shoots are transferred to rooting media or grafted. Rooted plantlets are transferred to the greenhouse after an acclimation period. Plants containing the transgene are then sprayed with HPPD inhibitor herbicides, as for example with tembotrione at a rate of 100 g AI/ha or with mesotrione at a rate of 300 g AI/ha supplemented with ammonium sulfate methyl ester rapeseed oil. Ten days after the application the symptoms due to the application of the herbicide are evaluated and compared to the symptoms observed on wild type plants under the same conditions.

### Example 3. Transformation of Maize Cells with the Expression Construct(s) Described Herein

Maize ears are best collected 8-12 days after pollination. Embryos are isolated from the ears, and those embryos 0.8-1.5 mm in size are preferred for use in transformation. Embryos are plated scutellum side-up on a suitable incubation media, such as DN62A5S media (3.98 g/L N6 Salts; 1 mL/L (of 1000× Stock) N6 Vitamins; 800 mg/L L-Asparagine; 100 mg/L Myo-inositol; 1.4 g/L L-Proline; 100 mg/L Casamino acids; 50 g/L sucrose; 1 mL/L (of 1 mg/mL Stock) 2,4-D). However, media and salts other than DN62A5S are suitable and are known in the art. Embryos are incubated overnight at 25° C. in the dark. However, it is not necessary per se to incubate the embryos overnight.

The resulting explants are transferred to mesh squares (30-40 per plate), transferred onto osmotic media for about 30-45 minutes, then transferred to a beaming plate (see, for example, PCT Publication No. WO/0138514 and U.S. Pat. No. 5,240,842).

DNA constructs designed to the genes of the invention in plant cells are accelerated into plant tissue using an aerosol beam accelerator, using conditions essentially as described in PCT Publication No. WO/0138514. After beaming, embryos are incubated for about 30 min on osmotic media, and placed onto incubation media overnight at 25° C. in the dark. To avoid unduly damaging beamed explants, they are incubated for at least 24 hours prior to transfer to recovery media. Embryos are then spread onto recovery period media, for about 5 days, 25° C. in the dark, then transferred to a selection media. Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated by methods known in the art. The resulting shoots are allowed to root on rooting media, and the resulting plants are transferred to nursery pots and propagated as transgenic plants.

TABLE 1
Materials, DN62A5S Media

| Components | Per Liter | | Source |
|---|---|---|---|
| Chu's N6 | 3.98 | g/L | Phytotechnology Labs |
| Basal Salt Mixture | | | |
| (Prod. No. C 416) | | | |
| Chu's N6 | 1 mL/L (of 1000x Stock) | | Phytotechnology Labs |
| Vitamin Solution | | | |
| (Prod. No. C 149) | | | |
| L-Asparagine | 800 | mg/L | Phytotechnology Labs |
| Myo-inositol | 100 | mg/L | Sigma |
| L-Proline | 1.4 | g/L | Phytotechnology Labs |
| Casamino acids | 100 | mg/L | Fisher Scientific |
| Sucrose | 50 | g/L | Phytotechnology Labs |

| | | |
|---|---|---|
| 2,4-D (Prod. | 1 mL/L (of 1 mg/mL Stock) | Sigma |
| No. D-7299) | | |

The pH of the solution is adjusted to pH 5.8 with IN KOH/IN KCl, Gelrite (Sigma) is added at a concentration up to 3 g/L, and the media is autoclaved. After cooling to 50° C., 2 ml/L of a 5 mg/ml stock solution of silver nitrate (Phytotechnology Labs) is added.

### Example 4. Transformation of Genes of the Invention in Plant Cells by Agrobacterium-Mediated Transformation

Ears are best collected 8-12 days after pollination. Embryos are isolated from the ears, and those embryos 0.8-1.5 mm in size are preferred for use in transformation. Embryos are plated scutellum side-up on a suitable incubation media, and incubated overnight at 25° C. in the dark. However, it is not necessary per se to incubate the embryos overnight. Embryos are contacted with an Agrobacterium strain containing the appropriate vectors for Ti plasmid mediated transfer for about 5-10 min, and then plated onto co-cultivation media for about 3 days (22° C. in the dark). After co-cultivation, explants are transferred to recovery period media for 5-10 days (at 25° C. in the dark). Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated as known in the art.

What is claimed is:

1. A transcription regulating nucleotide sequence that confers constitutive gene expression in a plant, wherein said transcription regulating nucleotide sequence has been modified by deleting at least one occurrence of the Motif from its DNA sequence.

2. The transcription regulating nucleotide sequence of claim 1, wherein the transcription regulating nucleotide sequence confers constitutive gene expression in either a monocotyledonous or a dicotyledonous plant.

3. The transcription regulating nucleotide sequence of claim 1, wherein two, three, or more occurrences of the Motif have been deleted.

4. The transcription regulating nucleotide sequence of claim 2, wherein the dicotyledonous plant is a soybean plant.

5. (canceled)

6. (canceled)

7. (canceled)

8. The transcription regulating nucleotide sequence of claim 1, wherein the transcription regulating nucleotide sequence is a DNA sequence having at least 60%, 65%, 70%, 75%, 80%, 85%, 90%, 95%, 99%, 100% sequence identity to any one of SEQ ID NOs: 1, 4 or 6 or a functional fragment thereof.

9. An expression cassette comprising the transcription regulating nucleotide sequence of claim 1.

10. A plant cell comprising the expression cassette of claim 9.

11. (canceled)

12. (canceled)

13. An expression cassette for regulating constitutive expression of a polynucleotide of interest, said expression cassette comprising a transcription regulating nucleotide sequence having at least 60%, 65%, 70%, 75%, 80%, 85%, 90%, 95%, 99%, 100% sequence identity to any one of the nucleic acid sequences set forth in SEQ ID NO: 1, SEQ ID NO: 2, SEQ ID NO: 3, SEQ ID NO: 4, SEQ ID NO: 5, SEQ ID NO: 6, or a functional fragment thereof.

14. (canceled)

15. The expression cassette of claim 13, wherein said expression cassette further comprises at least one polynucleotide of interest being operatively linked to the transcription regulating nucleotide sequence.

16. The expression cassette of claim 9, wherein the polynucleotide of interest encodes an insecticidal protein or an herbicidal selectable marker.

17. (canceled)

18. (canceled)

19. (canceled)

20. (canceled)

21. (canceled)

22. (canceled)

23. (canceled)

24. (canceled)

25. A transgenic plant tissue, plant organ, plant or seed comprising the expression cassette of claim 9.

26. (canceled)

27. (canceled)

28. (canceled)

29. (canceled)

30. (canceled)

31. (canceled)

32. (canceled)

33. A method for producing a transgenic plant tissue, plant organ, plant or seed comprising
(a) introducing the expression cassette of claim 9 into a plant cell; and
(b) regenerating said plant cell to form a plant tissue, plant organ, plant or seed.

34. (canceled)

35. (canceled)

36. (canceled)

37. (canceled)

38. (canceled)

39. A method of identifying a promoter, the method comprising the steps of:
(a) analyze RNA sequence expression datasets across tissue types from a plant species;
(b) identify at least one k-mer associated with the desired gene expression; (c) identify a CDS having said at least one k-mer upstream of said CDS; (d) select at least 100 bp, 200 bp, 300 bp, 400 bp, 500 bp, 600 bp, 700 bp, 800 bp, 900 bp or greater than or equal to 1000 bp, 1500 bp, or 2000 bp nucleotide sequence upstream from said CDS: thereby identifying a promoter sequence.

40. The method of claim 39, wherein the plant species is a dicotyledonous or monocotyledonous plant species.

41. The method of claim 40, wherein the plant species is a soybean plant.

42. (canceled)

43. (canceled)

44. (canceled)

45. (canceled)

46. (canceled)

47. (canceled)

48. (canceled)

49. A promoter that has been modified from its native form by (a) deleting at least one occurrence of the sequence GATCTG; or (b) modifying the Motif sequence wherein such deletion or modification results in promoter having a decreased expression variance as compared to the same promoter that does not contain said deletion or modification.

50. The promoter of claim 49, wherein said promoter is a dicotyledonous promoter.

51. The promoter of claim 50, wherein said promoter is a soybean promoter

52. The promoter of claim 49, wherein said promoter is a constitutive promoter.

53. The promoter of claim 52, wherein said promoter has decreased expression variance as compared to its native form.

54. A method of making a promoter with decreased expression variance, the method comprising the steps of: (a) analyze RNA sequence expression datasets across tissue types from a plant species; (b) identify at least one k-mer associated with the desired gene expression; (c) identify a CDS having said at least one k-mer upstream of said CDS; (d) select at least 100 bp, 200 bp, 300 bp, 400 bp, 500 bp, 600 bp, 700 bp, 800 bp, 900 bp or greater than or equal to 1000 bp nucleotide sequence upstream from said CDS; (e) remove or modify the sequence of at least one instance of said at least one k-mer identified in step (b); thereby making a promoter with decrease expression variance.

55. The method of claim 54, wherein the plant species is a dicotyledonous or monocotyledonous plant species.

56. (canceled)

57. (canceled)

58. (canceled)

59. The method of claim 54, wherein the at least one k-mer is GATCTG.

60. A promoter produced using the method of claim 54.

61. A plant cell, plant or plant part comprising the promoter of claim 60.

62. A vector comprising the promoter of claim 60.

63. A bacterial cell comprising the promoter of claim 60.

64. A expression cassette comprising a soybean constitutive promoter having at least one instance of the k-mer GATCTG.

65. A plant expression vector comprising a promoter operably linked to a gene of interest having at least one instance of the k-mer GATCTG.

66. A soybean promoter whose native form has been modified by editing or deleting at least one, at least 2, or at least 3, or more instances of the k-mer GATCTG.


SEQUENCE LISTING (6 sequences)

SEQ ID NO: 1 - DNA, 982 bp, synthetic construct
  source 1..982: mol_type = other DNA
  misc_feature 1..982: note = pUbi mutant
atatagaacacagagaatctcatttcttagaaataacgcagagtaggcccccactcaatgccacactccatttcacacacaaataaaaattaattttcttttacaaatattattaattattaggaaaaaacagacacttggtaactacctatcttttcatgtttttatcagatttgtaaaaataattataaaaataaaataataatttataaatgtaattacagaaataaaatagcataaaaatatgtacactagtaattcatcttcttgactatttcttatatttagttatagatataaaaaaatgtattccgcgtaaataaataaataaattaaattaaattcacttaagtccgattaatttacttaaaaaattaatttaataaataataaatttctacaatttattaccatgaattgtacagttaaattactacttaatttcatattcaaatccttaaggaataaattgagttaaatttaaataaatatggtaaagaaacagtctcttttcaaaggtgtagttaagttaacctcatttctggctggcttcctggtggacccttgcaggataccctcaatgacgagtcattcttggaaccaataaataataaataaataattacataaaataataacaataattaattattaattaattaattatagaagaataacccaaacccaatcaatcacagagaagaagatcgattccgaagccccacataccatttgttattcatataaaaactcgttttccttctccatttctcgtcgcaccttttcctcttctctctctttctctctttctagttcgtgttcttcgtcgcaaatccgaagtaggatctctcttgttttttctctaggtatgcaaatatttgtttatttatttatttattataggattatttcttagcctacgcgtgttttgttcgtgttttgcgttttagccgagttgtggtttcttgttaggtttttcgcg

SEQ ID NO: 2 - DNA, 1000 bp, Glycine max
  source 1..1000: mol_type = genomic DNA
  misc_feature 1..1000: note = pUbi native
atatagaacacagagaatctcatttcttagaaataacgcagagtaggcccccactcaatgccacactccatttcacacacaaataaaaattaattttcttttacaaatattattaattattaggaaaaaacagacacttggtaactacctatcttttcatgtttttatcagatttgtaaaaataattataaaaataaaataataatttataaatgtaattacagaaataaaatagcataaaaatatgtacactagtaattcatcttcttgactatttcttatatttagttatagatataaaaaaatgtattccgcgtaaataaataaataaattaaattaaattcacttaagtccgattaatttacttaaaaaattaatttaataaataataaatttctacaatttattaccatgaattgtacagttaaattactacttaatttcatattcaaatccttaaggaataaattgagttaaatttaaataaatatggtaaagaaacagtctcttttcaaaggtgtagttaagttaacctcatttctggctggcttcctggtggacccttgcaggataccctcaatgacgagtcattcttggaaccaataaataataaataaataattacataaaataataacaataattaattattaattaattaattatagaagaataacccaaacccaatcaatcacagagaagaagatcgattccgaagccccacataccatttgttattcatataaaaactcgttttccttctccatttctcgtcgcaccttttcctcttctctctctttctctctttctagttcgtgttcttcgtcgcaaatccgaagtaggatctctcttgttttttctctaggtatgcaaatatttgtttatttatttatttattataggattatttcttagcctacgcgtgttttggatctgttcgtgttttgcgttttgatctgagccgagttgtggtttcgatctgttgttaggtttttcgcg

SEQ ID NO: 3 - DNA, 1000 bp, Glycine max
  source 1..1000: mol_type = genomic DNA
  misc_feature 1..1000: note = P-tmn12-1.1 glyma.13G026100 native
tgaattaaaatcattaattattagaaaacttaatttataattaacttaataattttattaattaataataacaaatgaattaaaattattaatttataaaattaggaatcaaatcttaccgctaaaaaaaatcaaaatggtgattaggaaaccaaaatagtaacttaatcatattaataaataaggtcagatgcacataattatttgggaggcgtaggtgggctatccatacaaacatgggaatatcatgcccaacccgatgccattattgttcgctacatttctgattctttctcgctcacacttgcgacaccttcaacacaagaacaataatcatctgtggcgttgcgatatctctccctttcccaaactcaatatatcgccaaaattcctgggtgggtttctccgttctctctctgtctctctgatagcggagaaactacgtcttacttagatctagcggttttttcgctcttcttctgctccttgtttgtttgggttttattaagtggttggacacatgtgtgtttattgttctcgcggcctttaatttcagcaactttggatccctgtggctgttgtattcttgaattgttttgtgggatattttttttatttaatgttattattattactatattggatggtttgatttttgtcgttgccgttcaccgtactattagtacggcttgtgggtggaatccataactttattttacgatctgcctcaggctttttcataaaaatgatctgtgtttatttttgttcacttgtggatctgatttgaaccctgtgatgacatatgcttcagggtttggttatatcttttatatgcttttattatctttggcatgtgatgttgaattttgttttactcttttgtctagatttggtattgactgcaccggtgttggtgttggtggttgggagttagcttattagaagaagattcacagagaagtttttctgcatctgctgttgttgtattgcattgaaggaa

SEQ ID NO: 4 - DNA, 982 bp, synthetic construct
  source 1..982: mol_type = other DNA
  misc_feature 1..982: note = P-tmn12-1.1 glyma.13G026100 mutant
tgaattaaaatcattaattattagaaaacttaatttataattaacttaataattttattaattaataataacaaatgaattaaaattattaatttataaaattaggaatcaaatcttaccgctaaaaaaaatcaaaatggtgattaggaaaccaaaatagtaacttaatcatattaataaataaggtcagatgcacataattatttgggaggcgtaggtgggctatccatacaaacatgggaatatcatgcccaacccgatgccattattgttcgctacatttctgattctttctcgctcacacttgcgacaccttcaacacaagaacaataatcatctgtggcgttgcgatatctctccctttcccaaactcaatatatcgccaaaattcctgggtgggtttctccgttctctctctgtctctctgatagcggagaaactacgtcttacttagatctagcggttttttcgctcttcttctgctccttgtttgtttgggttttattaagtggttggacacatgtgtgtttattgttctcgcggcctttaatttcagcaactttggatccctgtggctgttgtattcttgaattgttttgtgggatattttttttatttaatgttattattattactatattggatggtttgatttttgtcgttgccgttcaccgtactattagtacggcttgtgggtggaatccataactttattttaccctcaggctttttcataaaaattgtttatttttgttcacttgtgatttgaaccctgtgatgacatatgcttcagggtttggttatatcttttatatgcttttattatctttggcatgtgatgttgaattttgttttactcttttgtctagatttggtattgactgcaccggtgttggtgttggtggttgggagttagcttattagaagaagattcacagagaagtttttctgcatctgctgttgttgtattgcattgaaggaa

SEQ ID NO: 5 - DNA, 999 bp, Glycine max
  source 1..999: mol_type = genomic DNA
  misc_feature 1..999: note = P-csi1-1.1 glyma.18G019600 native
aaaagattataagtattgattattatctactctgaaatgaaatagattggggtggaattagaagagaaaataataataaaaatgataagtgtactgatgagaaaaaaatttaaaaaagaaaaatgatgagatacaaatttgtccatgcgagaaatgatataaaaatgtaaaaagaggtacattattttatttccacgtgattctattccaaacgggtggctcaagcgaacctgaaggctgagcctagccgttaaatgacgcggcgaggctatgagcttgcaggctggagctgttgtcggtgctcattgtcacaaacactcccttctccgtcaattcaaaaccgaaaaatcacataacgcactcttttcttaacgaagcctcacctctaataatcaataatcaaatctgtctcccactaccaccaccgccaccgctaaaccaatccatgcgtcattctcacaccaacaaacctgcttcgcttctcactccctcaaggtctctctcacctcttcgttttttttttcaattcttcaattttcactagcattcattcgattcctgcattccgtcgtaaccgctttgactttttcgttgaccccgctgcacgctttccactcccattcctcggtagtagctgttgatctgctacctccaatctctctgactgtctctcgcgcgattcaattttaaccgattccattgttagcttgatttttttttttacagtgttttggttaggtcactgtgtagtttttaacttgttgaacttgcatttgcgatttctgtgcaattgctgtttggtttctgatctgaattaccgtgtggtgtggacagggaggctttgatgtgctgacgaactctccagtgtagatctggagagggagcagtgtcctggattgatctgcttggaggagaattcgagtgacgcggagcgaagctggcgactacactgacttggaggtttgctgccaataatggcagcacccttgctgctaatga

SEQ ID NO: 6 - DNA, 975 bp, synthetic construct
  source 1..975: mol_type = other DNA
  misc_feature 1..975: note = P-csi1-1.2 glyma.18G019600 mutant
aaaagattataagtattgattattatctactctgaaatgaaatagattggggtggaattagaagagaaaataataataaaaatgataagtgtactgatgagaaaaaaatttaaaaaagaaaaatgatgagatacaaatttgtccatgcgagaaatgatataaaaatgtaaaaagaggtacattattttatttccacgtgattctattccaaacgggtggctcaagcgaacctgaaggctgagcctagccgttaaatgacgcggcgaggctatgagcttgcaggctggagctgttgtcggtgctcattgtcacaaacactcccttctccgtcaattcaaaaccgaaaaatcacataacgcactcttttcttaacgaagcctcacctctaataatcaataatcaaatctgtctcccactaccaccaccgccaccgctaaaccaatccatgcgtcattctcacaccaacaaacctgcttcgcttctcactccctcaaggtctctctcacctcttcgttttttttttcaattcttcaattttcactagcattcattcgattcctgcattccgtcgtaaccgctttgactttttcgttgaccccgctgcacgctttccactcccattcctcggtagtagctgttctacctccaatctctctgactgtctctcgcgcgattcaattttaaccgattccattgttagcttgatttttttttttacagtgttttggttaggtcactgtgtagtttttaacttgttgaacttgcatttgcgatttctgtgcaattgctgtttggtttctaattaccgtgtggtgtggacagggaggctttgatgtgctgacgaactctccagtgtagagagggagcagtgtcctggattcttggaggagaattcgagtgacgcggagcgaagctggcgactacactgacttggaggtttgctgccaataatggcagcacccttgctgctaatga